(19) United States
(12) Patent Application Publication
(10) Pub. No.: US 2019/0173021 A1
(43) Pub. Date: Jun. 6, 2019

(54) NITROGEN HETEROCYCLE-FUSED BENZENE-BENZIMIDAZOLE ORGANIC COMPOUND, DISPLAY PANEL AND DISPLAY APPARATUS

(71) Applicant: WUHAN TIANMA MICRO-ELECTRONICS CO., LTD., Wuhan (CN)

(72) Inventors: Lei ZHANG, Wuhan (CN); Wei GAO, Wuhan (CN); Qing ZHU, Wuhan (CN); Jinghua NIU, Wuhan (CN); Ying LIU, Wuhan (CN); Ping AN, Wuhan (CN)

(73) Assignee: WUHAN TIANMA MICRO-ELECTRONICS CO., LTD., Wuhan (CN)

(21) Appl. No.: 16/266,066

(22) Filed: Feb. 3, 2019

(30) Foreign Application Priority Data
Sep. 20, 2018 (CN) 201811099985.2

(51) Int. Cl. H01L 51/00; C07D 401/14; C07D 403/14; C09K 11/06

(52) U.S. Cl. CPC H01L 51/0072; C07D 401/14; H01L 51/5012; C09K 11/06; C09K 2211/1018; C07D 403/14

(57) ABSTRACT

The present disclosure relates to a field of organic electroluminescent materials, and in particular to a novel nitrogen heterocycle-fused benzene-benzimidazole organic compound, a display panel and a display apparatus. The nitrogen heterocycle-fused benzene-benzimidazole organic compound has a structure represented by the following formula. The compound according to the present disclosure can be applied to a capping layer, a hole transmission layer, an electron transmission layer and a light-emitting layer of an organic light-emitting device, which can improve light extraction efficiency and luminous efficiency of a top-emission type organic photoelectric device (especially most effective for pixels of blue light), and can alleviate the angular dependence of luminescence of OLED device (most effective for pixels of red/green light), while effectively blocking water and oxygen in the external environment, and protecting the OLED display panel from erosion of water and oxygen.

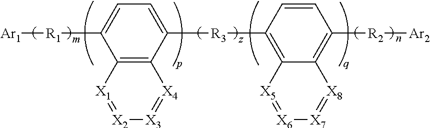

### CROSS-REFERENCE TO RELATED APPLICATIONS

The present disclosure claims priority to Chinese Patent Application No. 201811099985.2, filed on Sep. 20, 2018, the content of which is incorporated herein by reference in its entirety.

### FIELD

The present disclosure relates to the field of organic electroluminescent materials, and in particular, to a novel nitrogen heterocycle-fused benzene-benzimidazole organic compound, and a display panel and display apparatus containing the nitrogen heterocycle-fused benzene-benzimidazole organic compound.

### BACKGROUND

Organic light-emitting diode (OLED) has made great progress after several decades of development. Although an internal quantum efficiency of the OLED is already close to 100%, an external quantum efficiency of the OLED is only about 20%. Most of light are confined inside the light-emitting device due to factors such as surface plasma loss and waveguide effect, resulting in a large energy loss.

In a top-emission type device, an organic capping layer (CPL) is usually deposited on a translucent metal electrode Al to adjust an optical interference distance, suppress external light reflection, and suppress extinction caused by movement of surface plasmas, thereby enhancing extraction efficiency of the light, and improving luminous efficiency.

The existing CPL materials mostly use aromatic amine derivatives, phosphoxy derivatives and quinolinone derivatives, etc., which have both hole transmission function and electron transmission function and then the light extraction efficiency is improved to some extent. However, the refractive index of the existing CPL materials is generally no greater than 1.9, which does not meet the requirements of high refractive index; an amine derivative of specific structures having high refractive index and materials conforming to specific parameters can improve the light extraction efficiency, but cannot simultaneously solve the problem of luminous efficiency, especially for blue light-emitting elements. In order to increase the density of molecules and achieve high thermal stability, molecular structures of the existing materials are designed too big and loose, and molecules cannot be closely packed, resulting too many molecular gel holes during evaporation and poor coverage tightness.

### SUMMARY

The present disclosure provides a series of novel nitrogen heterocycle-fused benzene-benzimidazole organic compounds in which a nitrogen heterocycle-fused benzene-benzimidazole structure is used as a central skeleton.

In one embodiment, the present disclosure provides a nitrogen heterocycle-fused benzene-benzimidazole organic compound, having a chemical structure represented by chemical formula I:

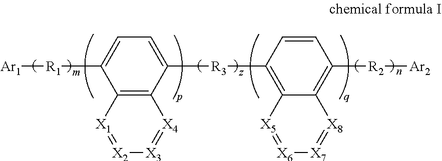

each of X₁-X₈ independently represents a nitrogen atom or a carbon atom, and at least one of X₁-X₄ is a nitrogen atom, and at least one of X₅-X₈ is a nitrogen atom; m, n, p, q, and z each independently represent a number of 0, 1, or 2, and p+q≥1;

R₁, R₂, and R₃ each independently represent a single bond, substituted or unsubstituted C1-C30 alkyl, substituted or unsubstituted silicylene, substituted or unsubstituted C1-C30 alkoxy, substituted or unsubstituted C6-C40 aryl, substituted or unsubstituted C10-C40 fused aryl, or substituted or unsubstituted C6-C40 heteroaryl;

Ar₁ and Ar₂ each independently represent one of the following structures shown by chemical formula II, chemical formula III, and chemical formula IV:

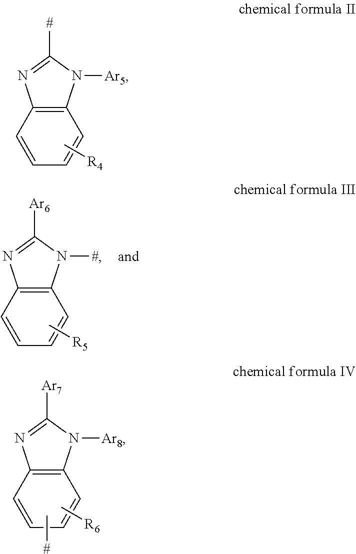

# represents a bonding position in the chemical formula I;

Ar₅, Ar₆ and Ar₇ each are independently selected from a group consisting of hydrogen atom, substituted or unsubstituted C1-C30 alkyl, substituted or unsubstituted silicylene, substituted or unsubstituted C1-C30 alkoxy, substituted or unsubstituted C6-C40 aryl, substituted or unsubstituted C10-C40 fused aryl, and substituted or unsubstituted C6-C40 heteroaryl; and

R₄, R₅, and R₆ each are independently selected from a group consisting of hydrogen atom, substituted or unsubstituted C1-C30 alkyl, substituted or unsubstituted silicylene, substituted or unsubstituted C1-C30 alkoxy, substituted or unsubstituted C6-C40 aryl, substituted or unsubstituted C10-C40 fused aryl, and substituted or unsubstituted C6-C40 heteroaryl.

In another embodiment, the present disclosure provides a display panel including an organic light-emitting device, and the organic light-emitting device comprises an anode, a cathode arranged opposite to the anode, a capping layer located at a side of the cathode facing away from the anode, and an organic layer located between the anode and the cathode, and the organic layer comprises a hole transmission layer, an electron transmission layer, and a light-emitting layer, at least one of the capping layer, the hole transmission layer, the electron transmission layer, and the light-emitting layer is made of the nitrogen heterocycle-fused benzene-benzimidazole organic compound as described above.

In still another embodiment, the present disclosure provides a display apparatus including the above display panel.

### BRIEF DESCRIPTION OF DRAWINGS

Embodiments of the present disclosure or in the related art, and the accompanying drawings used in the embodiments or in the related art are briefly described below. The drawings described below are merely a part of embodiments of the present disclosure.

FIG. 1 is a chemical formula of a nitrogen heterocycle-fused benzene-benzimidazole organic compound according to an embodiment of the present disclosure.

FIG. 2 is a structural schematic diagram showing an organic light-emitting device according to an embodiment of the present disclosure.

FIG. 3 is a graph showing refractive index and extinction coefficient of a compound CP4 according to an embodiment of the present disclosure.

FIG. 4 is a graph showing refractive index and extinction coefficient of a compound CP11 according to an embodiment of the present disclosure.

FIG. 5 is a graph showing refractive index and extinction coefficient of comparative example Com-1 (CBP) in a comparative example.

FIG. 6 is a schematic diagram showing a display apparatus according to an embodiment of the present disclosure.

### DESCRIPTION OF EMBODIMENTS

The present disclosure is further described by the following embodiments and comparative examples, which are merely intended to illustrate but not to limit the present disclosure.

In one embodiment, the present disclosure provides a nitrogen heterocycle-fused benzene-benzimidazole organic compound, having a chemical structure represented by chemical formula I:

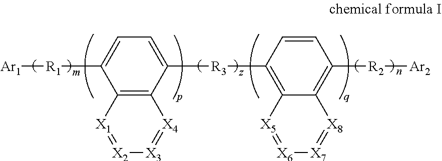

each of X₁-X₈ independently represents a nitrogen atom or a carbon atom, and at least one of X₁-X₄ is a nitrogen atom, and at least one of X₅-X₈ is a nitrogen atom; m, n, p, q, and z each independently represent a number of 0, 1, or 2, and p+q≥1;

R₁, R₂, and R₃ each independently represent a single bond, substituted or unsubstituted C1-C30 alkyl, substituted or unsubstituted silicylene, substituted or unsubstituted C1-C30 alkoxy, substituted or unsubstituted C6-C40 aryl, substituted or unsubstituted C10-C40 fused aryl, or substituted or unsubstituted C6-C40 heteroaryl;

Ar₁ and Ar₂ each independently represent one of the following structures shown by chemical formula II, chemical formula III, and chemical formula IV:

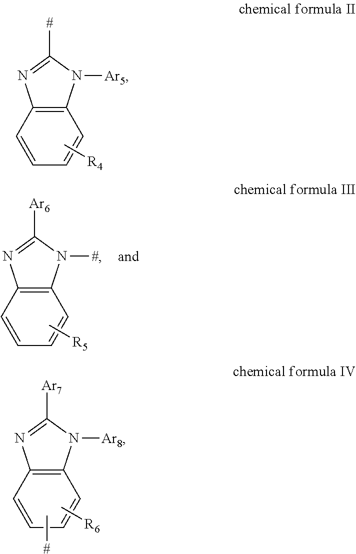

# represents a bonding position in the chemical formula I;

Ar₅, Ar₆ and Ar₇ each are independently selected from a group consisting of hydrogen atom, substituted or unsubstituted C1-C30 alkyl, substituted or unsubstituted silicylene, substituted or unsubstituted C1-C30 alkoxy, substituted or unsubstituted C6-C40 aryl, substituted or unsubstituted C10-C40 fused aryl, and substituted or unsubstituted C6-C40 heteroaryl; and

R₄, R₅, and R₆ each are independently selected from a group consisting of hydrogen atom, substituted or unsubstituted C1-C30 alkyl, substituted or unsubstituted silicylene, substituted or unsubstituted C1-C30 alkoxy, substituted or unsubstituted C6-C40 aryl, or substituted or unsubstituted C10-C40 fused aryl, and substituted or unsubstituted C6-C40 heteroaryl.

The nitrogen heterocycle-fused benzene-benzimidazole organic compound according to the present disclosure adopts an aromatic fused ring structure having a large density, so that molecular packing is better, and polarizability of fragments is larger under a reasonable design. For visible light having a wavelength between 400 nm and 700 nm, the nitrogen heterocycle-fused benzene-benzimidazole organic compound according to the present disclosure can provide a good refractive index (n≥2.0). For visible light having a wavelength between 430 nm and 700 nm, the extinction coefficient k of the nitrogen heterocycle-fused benzene-benzimidazole organic compound according to the present disclosure is less than or equal to 0. For visible light having a wavelength between 400 nm and 700 nm, transmittance of the nitrogen heterocycle-fused benzene-benzimidazole organic compound according to the present disclosure is greater than 65%. The nitrogen heterocycle-fused benzene-benzimidazole organic compound according to the present disclosure has a high glass transition temperature and good molecular thermal stability, i.e., no thermal decomposition occurs when evaporation is performed.

When the nitrogen heterocycle-fused benzene-benzimidazole organic compound according to the present disclosure is applied to an organic electroluminescent device, the light extraction efficiency and luminous efficiency of the top-emission type organic photoelectric device can be improved, especially most effective for improving luminous efficiency of blue pixels, and the angular dependence of light emission of the OLED device light can be alleviated, especially most effective for the red/green light pixel. Meanwhile, water and oxygen in the external environment can be effectively blocked, and the OLED display panel can be protected from erosion of water and oxygen.

According to an embodiment of the present disclosure, in the chemical formula I, m=n=0, p>0, q>0, and z>0.

According to an embodiment of the present disclosure, in the chemical formula I, q=z=0.

According to an embodiment of the present disclosure, in the chemical formula I, m>0, and n=0; or, n>0, and m=0.

According to an embodiment of the present disclosure, in the chemical formula I, p>0, q>0, and m=n=z=0.

According to an embodiment of the present disclosure, in the chemical formula I, p>0, and m=n=q=z=0.

According to an embodiment of the present disclosure, in the chemical formula I, m=n.

According to an embodiment of the present disclosure, in the chemical formula I, p>0, q>0, X₁ and X₈ represent a same atom, X₂ and X₇ represent a same atom, X₃ and X₆ represent a same atom, and X₄ and X₅ represent a same atom.

According to an embodiment of the present disclosure, in the chemical formula I, R₁ and R₂ represent a same substituent.

According to an embodiment of the present disclosure, in the chemical formula I, Ar₁ and Ar₂ represent a same substituent.

According to an embodiment of the present disclosure, in the chemical formula I, only one of X₁-X₄ represent a nitrogen atom, and/or only one of X₅-X₈ represent a nitrogen atom.

According to an embodiment of the present disclosure, in the chemical formula I, at least two of X₁-X₄ represent a nitrogen atom, and/or, at least two of X₅-X₈ represent a nitrogen atom.

According to an embodiment of the present disclosure, the nitrogen heterocycle-fused benzene-benzimidazole organic compound is one of the following compounds:

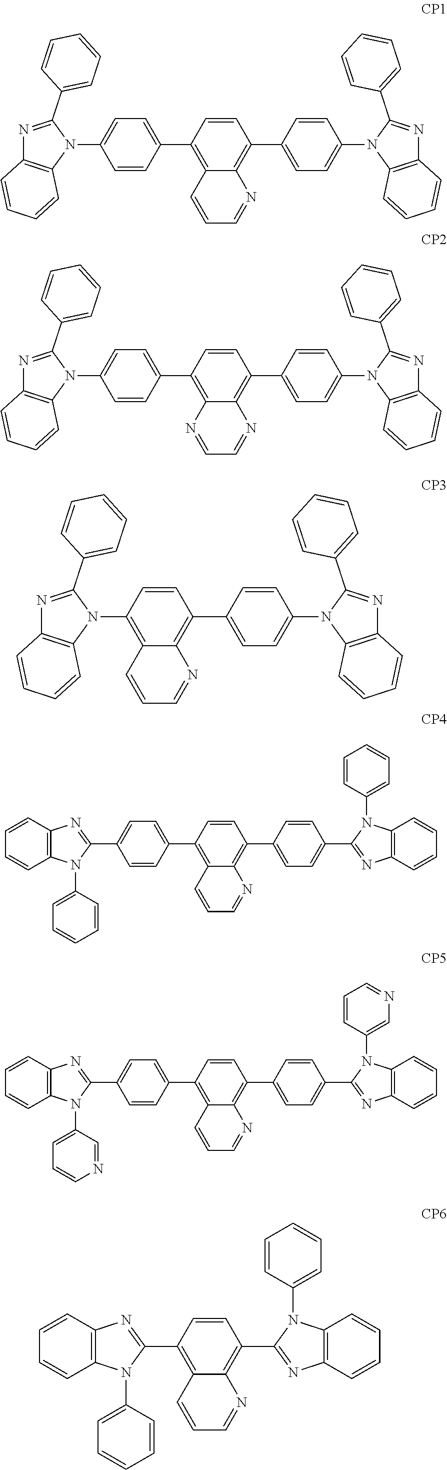

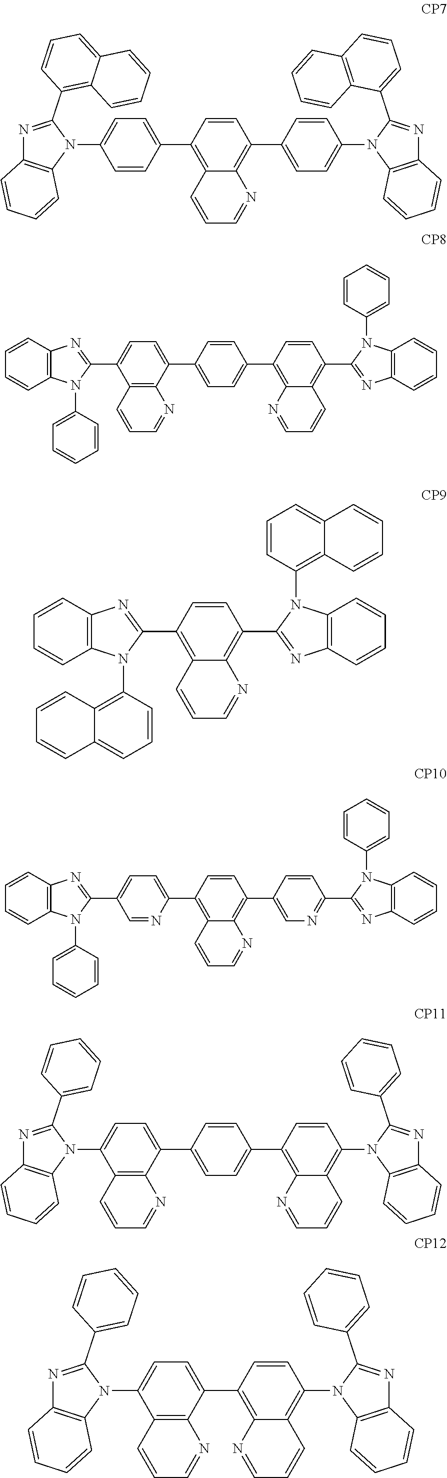

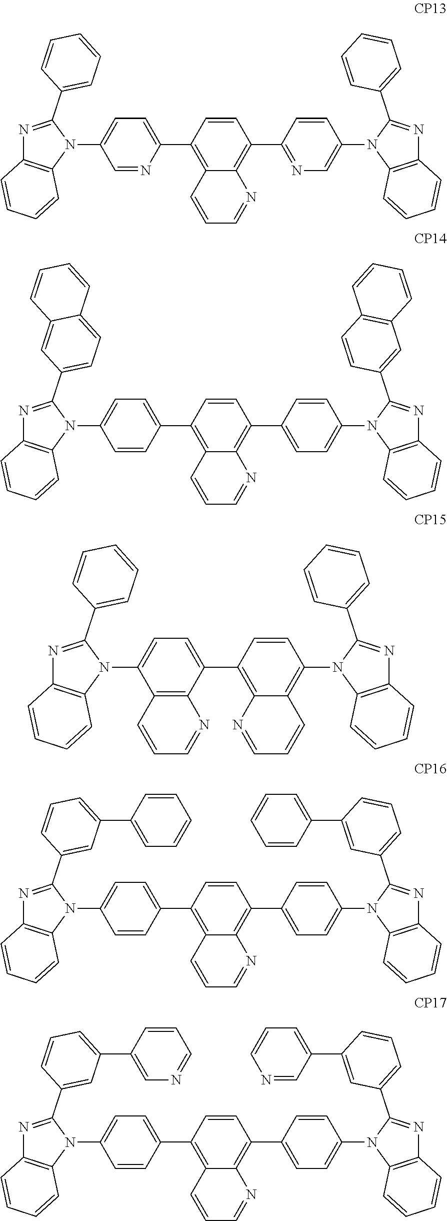

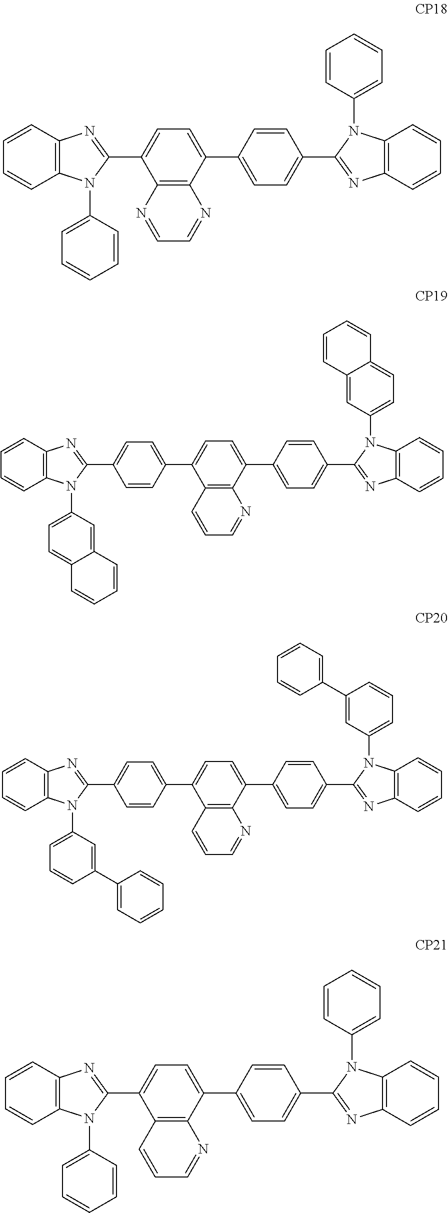 [SMILES: c1ccc(-n2c(-c3ccc(-c4ccc(-c5nc6ccccc6n5-c5ccccc5)c5nccnc45)cc3)nc3ccccc32)cc1 ; c1ccc2cc(-n3c(-c4ccc(-c5ccc(-c6ccc(-c7nc8ccccc8n7-c7ccc8ccccc8c7)cc6)c6ncccc56)cc4)nc4ccccc43)ccc2c1 ; c1ccc(-c2cccc(-n3c(-c4ccc(-c5ccc(-c6ccc(-c7nc8ccccc8n7-c7cccc(-c8ccccc8)c7)cc6)c6ncccc56)cc4)nc4ccccc43)c2)cc1 ; c1ccc(-n2c(-c3ccc(-c4ccc(-c5nc6ccccc6n5-c5ccccc5)c5cccnc45)cc3)nc3ccccc32)cc1]

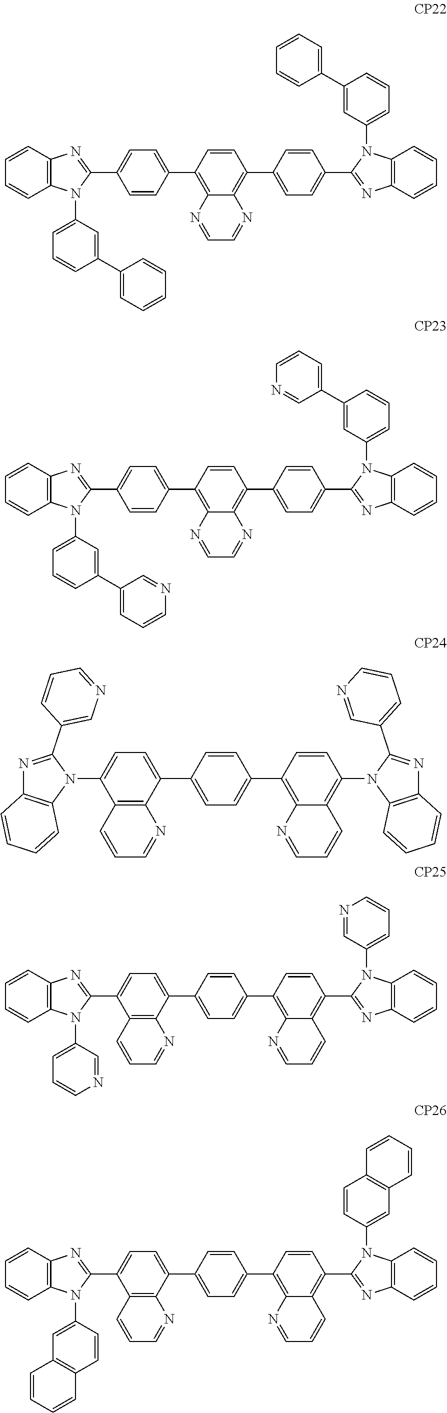

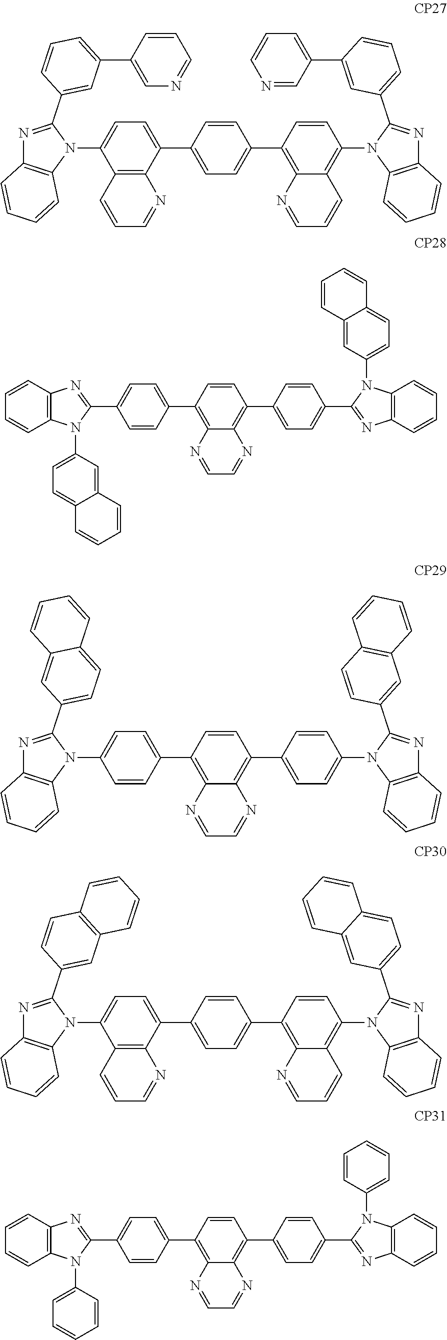

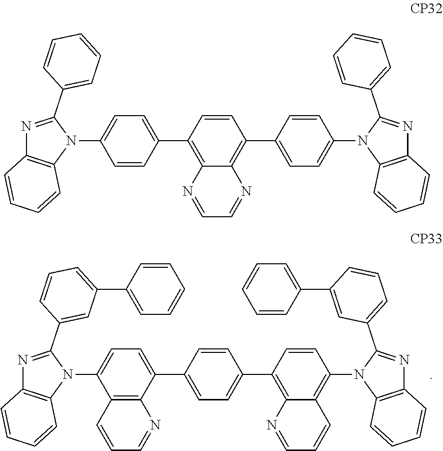 [SMILES: c1ccc(-c2nc3ccccc3n2-c2ccc(-c3ccc(-c4ccc(-n5c(-c6ccccc6)nc6ccccc65)cc4)c4nccnc34)cc2)cc1 ; c1ccc(-c2cccc(-c3nc4ccccc4n3-c3ccc(-c4ccc(-c5ccc(-n6c(-c7cccc(-c8ccccc8)c7)nc7ccccc76)c6cccnc56)cc4)c4ncccc34)c2)cc1]

According to an embodiment of the present disclosure, for visible light having a wavelength between 400 nm and 700 nm, the nitrogen heterocycle-fused benzene-benzimidazole organic compound has a refractive index n greater than or equal to 2.0.

According to an embodiment of the present disclosure, for visible light having a wavelength between 430 nm and 700 nm, the nitrogen heterocycle-fused benzene-benzimidazole organic compound has an extinction coefficient k less than or equal to 0.0.

According to an embodiment of the present disclosure, for visible light having a wavelength between 400 nm and 700 nm, the nitrogen heterocycle-fused benzene-benzimidazole organic compound has a transmittance greater than 65%.

The novel compounds with the above design can be applied to the capping layer CPL of an organic layer, and can serve as a host material, a doping material, or a material for an electron transmission layer or a hole transmission layer.

In another embodiment, the present disclosure provides an organic light-emitting display panel including an organic light-emitting device. The organic light-emitting device includes an anode, a cathode arranged opposite to the anode, a capping layer located at a side of the cathode facing away from the anode, an organic layer located between the anode and the cathode. The organic layer includes a hole transmission layer, an electron transmission layer, and a light-emitting layer. At least one of the capping layer, the hole transmission layer, the electron transmission layer, and the light-emitting layer is made of the nitrogen heterocycle-fused benzene-benzimidazole organic compound as described above.

In the organic light-emitting device provided by the present disclosure, the anode can be made of a metal selected from a group consisting of copper, gold, silver, iron, chromium, nickel, manganese, palladium, platinum, etc., and alloys thereof. The anode can also be made of metal oxide, such as indium oxide, zinc oxide, indium tin oxide (ITO), indium zinc oxide (IZO), and the like. The anode can also be made of a conductive polymer, such as polyaniline, polypyrrole, poly(3-methylthiophene) and the like. In addition to the anode material mentioned above, the anode can also be made of any suitable material or material combination including the suitable anode materials known in the related art, as long as the material is conducive to injecting holes.

In the organic light-emitting device provided by the present disclosure, the cathode can be made of metal, such as aluminum, magnesium, silver, indium, tin, titanium, etc., or alloys thereof. The cathode can also be made of multiple-layer metal material, such as LiF/Al, LiO₂/Al, BaF₂/Al, and the like. In addition to the cathode materials listed above, the cathode can also be made of any suitable material or material combination including the suitable cathode material known in the related art, as long as the material is conducive to injecting electrons.

According to embodiments of the present disclosure, the organic light-emitting device was manufactured by forming an anode on a transparent or opaque smooth substrate, forming an organic thin layer on the anode, and then forming a cathode on the organic thin layer. The organic thin layer can be formed by a known film-forming method such as vapor deposition, sputtering, spin coating, dipping, ion plating, and the like. Finally, an organic optical covering layer, i.e., a capping layer (CPL) was prepared on the cathode. The material of the CPL is the nitrogen heterocycle-fused benzene-benzimidazole organic compound described in the present disclosure. The CPL can be prepared by a vapor evaporation method or a solution method. The solution method includes an ink-jet printing method, spin coating, blade coating, screen printing, roll-to-roll printing, and the like.

The novel compounds with the above design can be applied to the capping layer CPL of the organic layer, and can also serve as a host material, a doping material, or a material for an electron transmission layer or a hole transmission layer.

Synthesis of intermediates M1-M7 is described below.

Synthesis of Intermediate M1

When one of X₁ and X₂ is a nitrogen atom, a synthetic route is as follows:

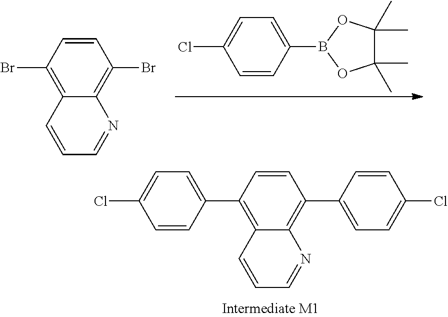 [SMILES: Brc1ccc(Br)c2ncccc12 ; CC1(C)OB(c2ccc(Cl)cc2)OC1(C)C ; Clc1ccc(-c2ccc(-c3ccc(Cl)cc3)c3ncccc23)cc1]

In nitrogen atmosphere, raw materials of 5,8-dibromoquinoline (0.012 mol), 1-chloro-4-phenylborate (0.025 mol) and palladium acetate (0.0003 mol) were sequentially added into 150 mL DMF in a 250 mL three-necked flask, mixed and stirred. After a solution of K₃PO₄ (0.045 mol) in water was added, the mixture was refluxed for 10 hours at a temperature of 130° C., and then naturally cooled to room temperature. After completion of the reaction, 100 mL deionized water was added, and then a few droplets of 2M HCl were added dropwise, and then the mixture was extracted with dichloromethane, and an organic phase was collected and dried by anhydrous Na₂SO₄. The dried solution was filtered, and the solvent was removed using a rotary evaporator to obtain a crude product. The obtained crude product was purified through silica gel column chromatography to obtain the Intermediate M1.

Elemental analysis results of the Intermediate M1 (Molecular Formula C₂₁H₁₃Cl₂N): theoretical value: C, 72.01; H, 3.74; Cl, 20.24; N, 4.40; test value: C, 72.03; H, 3.75; Cl, 20.24; N, 3.97. The ESI-MS (m/z) (M+) was obtained by liquid chromatography-mass spectrometry: theoretical value: 349.04, and test value: 349.25.

Synthesis of Intermediate M5

When one of X₁ and X₂ is a nitrogen atom, a synthetic route is as follows:

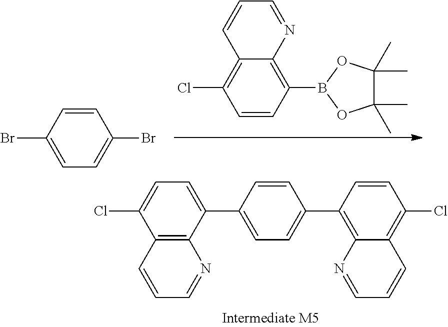

In nitrogen atmosphere, raw materials of 1,4-dibromobenzene (0.012 mol), 5-chloro-8-quinoline borate (0.025 mol) and palladium acetate (0.0003 mol) were sequentially added into 150 mL DMF in a 250 mL three-necked flask, mixed and stirred. After a solution of K₃PO₄ (0.045 mol) in water was added, the mixture was refluxed for 10 hours at a temperature of 130° C., and then naturally cooled to room temperature. After completion of the reaction, 100 mL deionized water was added, then a few droplets of 2M HCl were added, and then the mixture was extracted with dichloromethane, and an organic phase was collected and dried by anhydrous Na₂SO₄. The dried solution was filtered, and the solvent was removed using a rotary evaporator to obtain a crude product. The obtained crude product was purified through silica gel column chromatography to obtain the Intermediate M5.

Elemental analysis results of the Intermediate M5 (Molecular Formula C₂₄H₁₃Cl₂N₂): theoretical value: C, 71.83; H, 3.52; Cl, 17.67; N, 6.98; test value: C, 71.86; H, 3.53; Cl, 17.65; N, 6.96. The ESI-MS (m/z) (M+) was obtained by liquid chromatography-mass spectrometry: theoretical value: 400.05, and test value: 400.24.

Synthesis of Intermediate M7

When the Ar group is a phenyl group, a synthetic route is as follows:

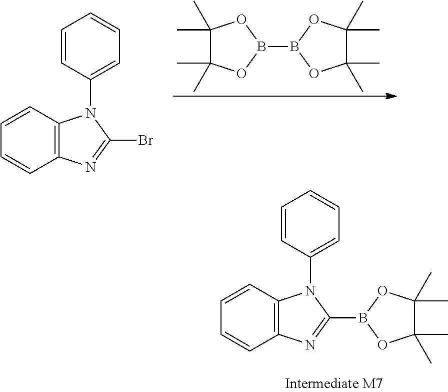

In nitrogen atmosphere, raw materials of 2-bromo-1-phenylbenzimidazole (0.012 mol), bis(pinacolato)diboron (0.012 mol) and palladium acetate (0.0003 mol) were sequentially added into 150 mL DMF in a 250 mL three-necked flask, mixed and stirred. After a solution of K₃PO₄ (0.045 mol) in water was added, the mixture was refluxed for 10 hours at a temperature of 130° C., and then naturally cooled to room temperature. After completion of the reaction, 100 mL deionized water was added, and then a few droplets of 2M HCl were added, and then the mixture was extracted with dichloromethane, and an organic phase was collected and dried by anhydrous Na₂SO₄. The dried solution was filtered, and the solvent was removed using a rotary evaporator to obtain a crude product. The obtained crude product was purified through silica gel column chromatography to obtain the Intermediate M7.

Elemental analysis results of the Intermediate M7 (Molecular Formula C₁₉H₂₁BN₂O₂): theoretical value: C, 71.27; H, 6.61; B, 3.38; N, 8.75; O, 9.99; test value: C, 71.28; H, 6.63; B, 3.38; N, 8.74; O, 9.97. The ESI-MS (m/z) (M+) was obtained by liquid chromatography-mass spectrometry: theoretical value: 320.17, and test value: 320.46.

Similarly, Intermediates M2, M3, M4, and M6 can be prepared. Structures of the Intermediates M1-M7 are shown in Table 1. In the following Intermediates, X₁ and X₂ are a C atom or a nitrogen atom, and at least one of X₁ and X₂ is a nitrogen atom; the Ar groups each are independently selected from a group consisting of hydrogen atom, substituted or unsubstituted C1-C30 alkyl, substituted or unsubstituted silicylene, substituted or unsubstituted C1-C30 alkoxy, substituted or unsubstituted C6-C40 aryl, substituted or unsubstituted C10-C40 fused aryl, and substituted or unsubstituted C6-C40 heteroaryl, which are consistent with the first aspect described above corresponding to the embodiments of the present disclosure.

TABLE 1
Structures of Intermediates M1-M7

| raw material 1 | raw material 2 | Intermediates |
|---|---|---|
| 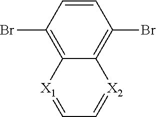 | 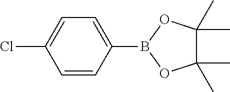 [SMILES: CC1(C)OB(c2ccc(Cl)cc2)OC1(C)C] | 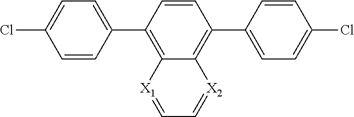 |
| 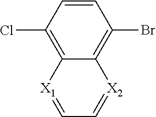 | 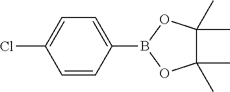 [SMILES: CC1(C)OB(c2ccc(Cl)cc2)OC1(C)C] | 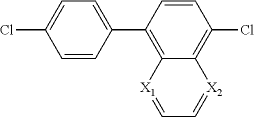 |
| NG | NG | 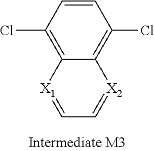 |
| 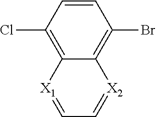 | 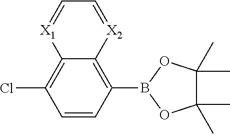 | 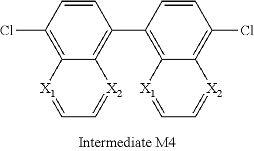 |
| 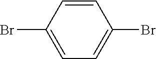 | 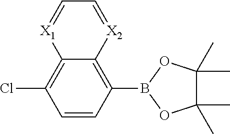 | 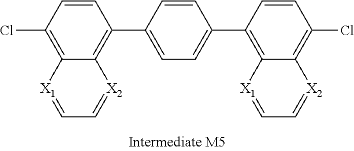 |
| 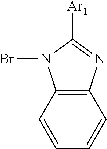 | 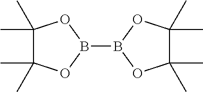 | 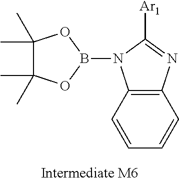 |
| 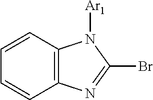 | 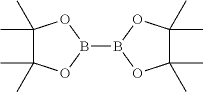 | 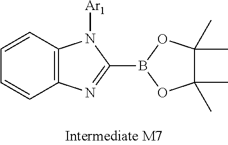 |

A further embodiment of the present disclosure provides methods for preparing several exemplary nitrogen heterocycle-fused benzene-benzimidazole organic compounds, which are described in exemplary Examples 1-6 below.

### EXAMPLE 1

A synthetic route of Compound CP1 is as follows:

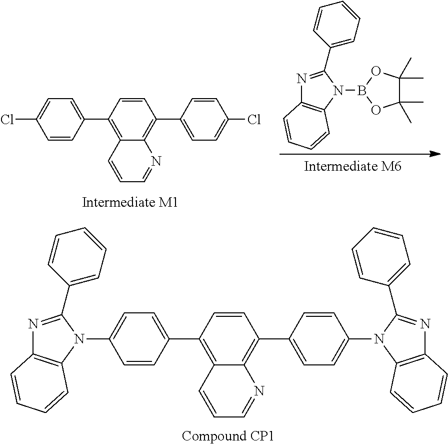

In nitrogen atmosphere, raw materials of the Intermediate M1 (0.012 mol), the Intermediate M6 (0.025 mol) and palladium acetate (0.0003 mol) were sequentially added into 150 mL DMF in a 250 mL three-necked flask, mixed and stirred. After a solution of K₃PO₄ (0.045 mol) in water was added, the mixture was refluxed for 10 hours at a temperature of 130° C., and then naturally cooled to room temperature. After completion of the reaction, 100 mL deionized water was added, and then a few droplets of 2M HCl were added, and then the mixture was extracted with dichloromethane, and an organic phase was collected and dried by anhydrous Na₂SO₄. The dried solution was filtered, and the solvent was removed using a rotary evaporator to obtain a crude product. The obtained crude product was purified through silica gel column chromatography to obtain the Compound CP1.

Elemental analysis results of the Compound CP1 (Molecular Formula C₄₇H₃₁N₅): theoretical value: C, 84.79; H, 4.69; N, 10.52; test value: C, 84.81; H, 4.70; N, 10.49. The ESI-MS (m/z) (M+) was obtained by liquid chromatography-mass spectrometry: theoretical value: 665.26, and test value: 665.50.

### EXAMPLE 2

A synthetic route of Compound CP4 is as follows:

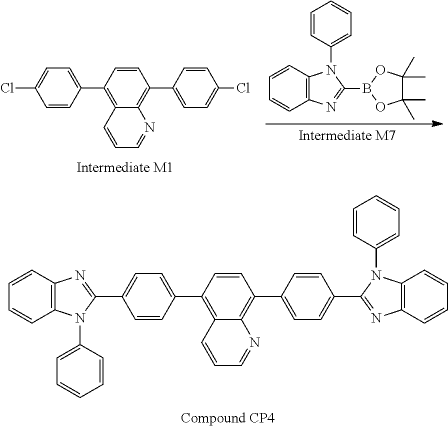

In nitrogen atmosphere, raw materials of the Intermediate M1 (0.012 mol), the Intermediate M7 (0.025 mol) and palladium acetate (0.0003 mol) were sequentially added into 150 mL DMF in a 250 mL three-necked flask, mixed and stirred. After a solution of K₃PO₄ (0.045 mol) in water was added, the mixture was refluxed for 10 hours at a temperature of 130° C., and then naturally cooled to room temperature. After completion of the reaction, 100 mL deionized water was added, and then a few droplets of 2M HCl were added, and then the mixture was extracted with dichloromethane, and an organic phase was collected and dried by anhydrous Na₂SO₄. The dried solution was filtered, and the solvent was removed using a rotary evaporator to obtain a crude product. The obtained crude product was purified through silica gel column chromatography to obtain the Compound CP4.

Elemental analysis results of the Compound CP4 (Molecular Formula C₄₇H₃₁N₅): theoretical value: C, 84.79; H, 4.69; N, 10.52; test value: C, 84.80; H, 4.71; N, 10.49. The ESI-MS (m/z) (M+) was obtained by liquid chromatography-mass spectrometry: theoretical value: 665.26, and test value: 665.30.

### EXAMPLE 3

A synthetic route of Compound CP6 is as follows:

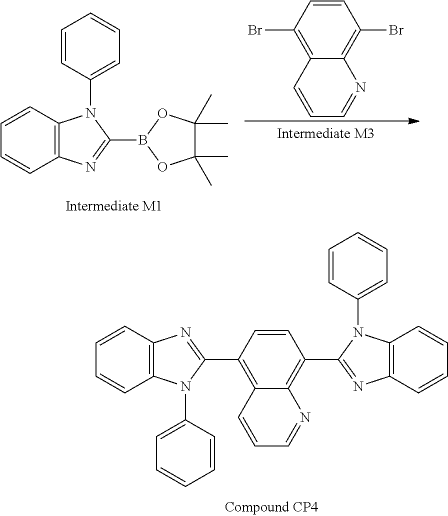 [SMILES: CC1(C)OB(c2nc3ccccc3n2-c2ccccc2)OC1(C)C ; Brc1ccc(Br)c2ncccc12 ; c1ccc(-n2c(-c3ccc(-c4nc5ccccc5n4-c4ccccc4)c4ncccc34)nc3ccccc32)cc1]

In nitrogen atmosphere, raw materials of the Intermediate M7 (0.025 mol), the Intermediate M3 (0.012 mol) and palladium acetate (0.0003 mol) were sequentially added to 150 mL DMF in a 250 mL three-necked flask, mixed and stirred. After a solution of K₃PO₄ (0.045 mol) in water was added, the mixture was refluxed for 10 hours at a temperature of 130° C., and then naturally cooled to room temperature. After completion of the reaction, 100 mL deionized water was added, and then a few droplets of 2M HCl were added, and then the mixture was extracted with dichloromethane, and an organic phase was collected and dried by anhydrous Na₂SO₄. The dried solution was filtered, and the solvent was removed using a rotary evaporator to obtain a crude product. The obtained crude product was purified through silica gel column chromatography to obtain the Compound CP6.

Elemental analysis results of the Compound CP6 (Molecular Formula C₃₅H₂₃N₅): theoretical value: C 81.85; H, 4.51; N, 13.64; test value: C, 81.83; H, 4.49; N, 13.68. The ESI-MS (m/z) (M+) was obtained by liquid chromatography-mass spectrometry: theoretical value: 513.20, and test value: 513.32.

### EXAMPLE 4

A synthetic route of Compound CP11 is as follows:

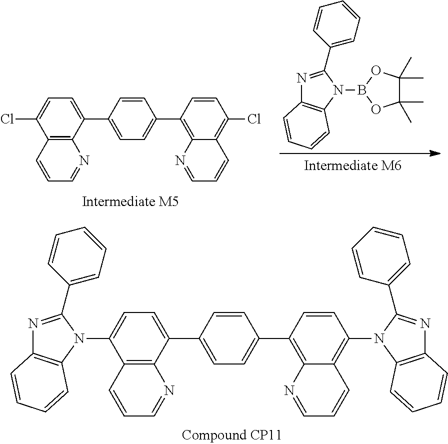

In nitrogen atmosphere, raw materials of the Intermediate M5 (0.012 mol), the Intermediate M6 (0.025 mol) and palladium acetate (0.0003 mol) were sequentially added to 150 mL DMF in a 250 mL three-necked flask, mixed and stirred. After a solution of K₃PO₄ (0.045 mol) in water was added, the mixture was refluxed for 10 hours at a temperature of 130° C., and then naturally cooled to room temperature. After completion of the reaction, 100 mL deionized water was added, and then a few droplets of 2M HCl were added, and then the mixture was extracted with dichloromethane, and an organic phase was collected and dried by anhydrous Na₂SO₄. The dried solution was filtered, and the solvent was removed using a rotary evaporator to obtain a crude product. The obtained crude product was purified through silica gel column chromatography to obtain the Compound CP11.

Elemental analysis results of the Compound CP11 (C₅₀H₃₂N₆): theoretical value: C, 83.78; H, 4.50; N, 11.72; test value: C, 83.76; H, 4.49; N, 11.75. The ESI-MS (m/z) (M+) was obtained by liquid chromatography-mass spectrometry: theoretical value: 716.27, and test value: 716.56.

### EXAMPLE 5

A synthetic route of Compound CP12 is as follows:

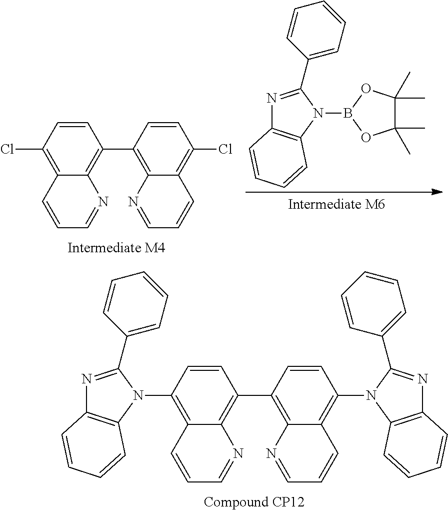

In nitrogen atmosphere, raw materials of the Intermediate M4 (0.012 mol), the Intermediate M6 (0.025 mol) and palladium acetate (0.0003 mol) were sequentially added to 150 mL DMF in a 250 mL three-necked flask, mixed and stirred. After a solution of K₃PO₄ (0.045 mol) in water was added, the mixture was refluxed for 10 hours at a temperature of 130° C., and then naturally cooled to room temperature. After completion of the reaction, 100 mL deionized water was added, and then a few droplets of 2M HCl were added, and then the mixture was extracted with dichloromethane, and an organic phase was collected and dried by anhydrous Na₂SO₄. The dried solution was filtered, and the solvent was removed using a rotary evaporator to obtain a crude product. The obtained crude product was purified through silica gel column chromatography to obtain the Compound CP12.

Elemental analysis results of the Compound CP12 (C₄₄H₂₈N₆): theoretical value: C, 82.48; H, 4.40; N, 13.12; test value: C, 82.50; H, 4.42; N, 13.08. The ESI-MS (m/z) (M+) was obtained by liquid chromatography-mass spectrometry: theoretical value: 640.24, and test value: 640.48.

### EXAMPLE 6

A synthetic route of Compound CP18 is as follows:

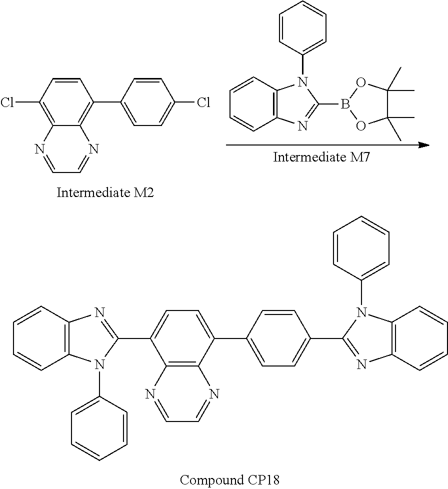

In nitrogen atmosphere, raw materials of the Intermediate M2 (0.012 mol), the Intermediate M7 (0.025 mol) and palladium acetate (0.0003 mol) were sequentially added to 150 mL DMF in a 250 mL three-necked flask, mixed and stirred. After a solution of K₃PO₄ (0.045 mol) in water was added, the mixture was refluxed for 10 hours at a temperature of 130° C., and then naturally cooled to room temperature. After completion of the reaction, 100 mL deionized water was added, and then a few droplets of 2M HCl were added, and then the mixture was extracted with dichloromethane, and an organic phase was collected and dried by anhydrous Na₂SO₄. The dried solution was filtered, and the solvent was removed using a rotary evaporator to obtain a crude product. The obtained crude product was purified through silica gel column chromatography to obtain the Compound CP18.

Elemental analysis results of the Compound CP18 (C₄₀H₂₆N₆): theoretical value: C, 81.34; H, 4.44; N, 14.23; test value: C, 81.34; H, 4.42; N, 14.25. The ESI-MS (m/z) (M+) was obtained by liquid chromatography-mass spectrometry: theoretical value: 590.22, and test value: 590.46.

Test results of the thermal properties and refractive indexes of the nitrogen heterocycle-fused benzene-benzimidazole organic compounds according to the present disclosure are listed in Table 2. In Table 2, the structural formulas of the compounds CBP, TPBI, and Alq3 used in Comparative Example Com-1, Comparative Example Com-2, and Comparative Example Com-3, respectively, are as follows:

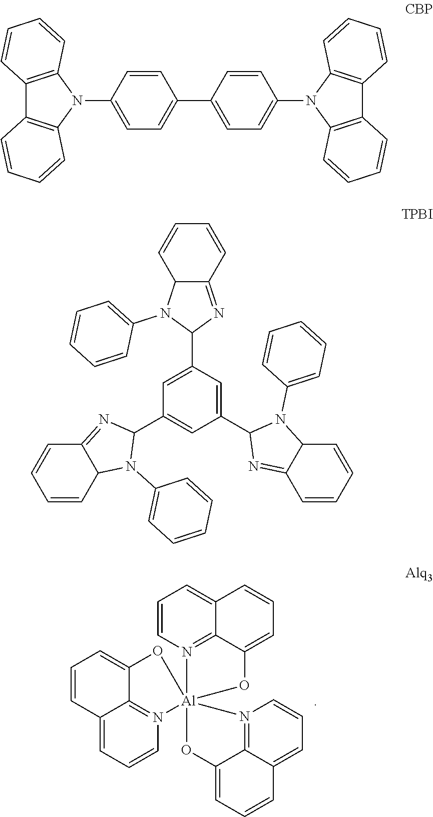

TABLE 2
test results of thermal performance and refractive index

| | | refractive index | | |
|---|---|---|---|---|
| compound | Tg (° C.) | n@450 | n@530 | n@620 |
| CP1 | 153 | 2.20 | 2.05 | 1.98 |
| CP4 | 160 | 2.35 | 2.14 | 2.05 |
| CP6 | 154 | 2.26 | 2.11 | 2.04 |
| CP11 | 157 | 2.30 | 2.09 | 2.01 |
| CP12 | 158 | 2.41 | 2.20 | 2.12 |
| CP15 | 156 | 2.34 | 2.13 | 2.06 |
| CP18 | 158 | 2.29 | 2.12 | 2.04 |
| CP23 | 153 | 2.26 | 2.11 | 2.04 |
| CP24 | 161 | 2.36 | 2.16 | 2.07 |
| CP27 | 158 | 2.34 | 2.15 | 2.08 |
| CP30 | 162 | 2.45 | 2.23 | 2.09 |
| CP32 | 155 | 2.39 | 2.18 | 2.07 |
| CBP | 108 | 1.87 | 1.81 | 1.78 |
| A1q3 | 149 | 1.78 | 1.75 | 1.73 |
| TPBI | 121 | 1.80 | 1.76 | 1.73 |

* Taking n@450 as an example, n@450 means the refractive index of the nitrogen heterocycle-fused benzene-benzimidazole compound according to the present disclosure at a wavelength of 450 nm.

As can be seen from Table 2 above, the nitrogen heterocycle-fused benzene-benzimidazole organic compounds provided by the present disclosure have a higher glass transition temperature Tg. For light having wavelengths of 450 nm, 530 nm, and 620 nm, all of the nitrogen heterocycle-fused benzene-benzimidazole organic compounds of the present disclosure have a refractive index greater than 2.0.

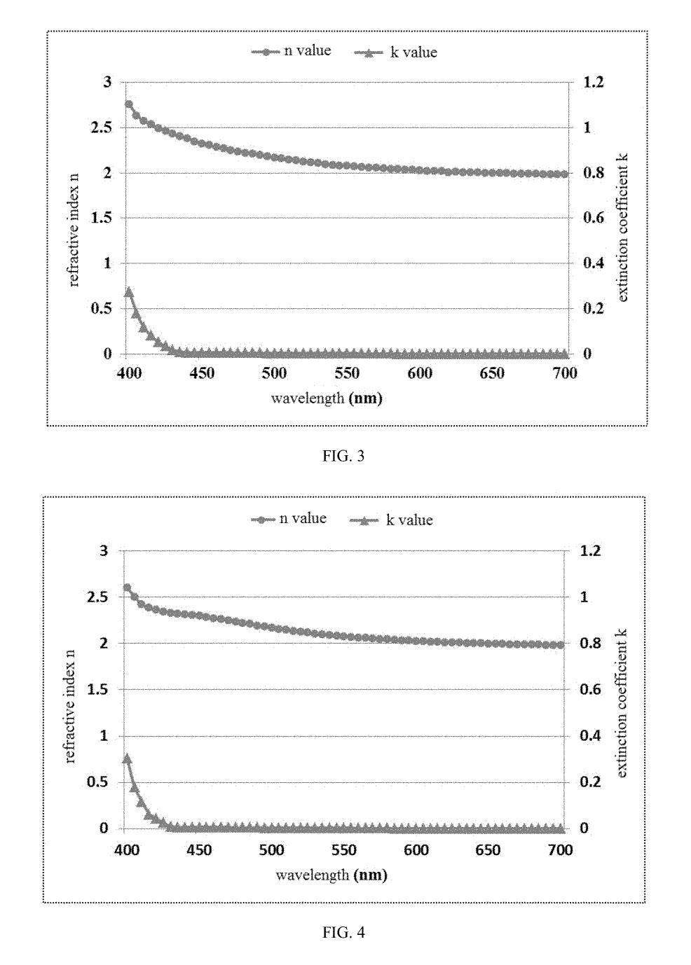

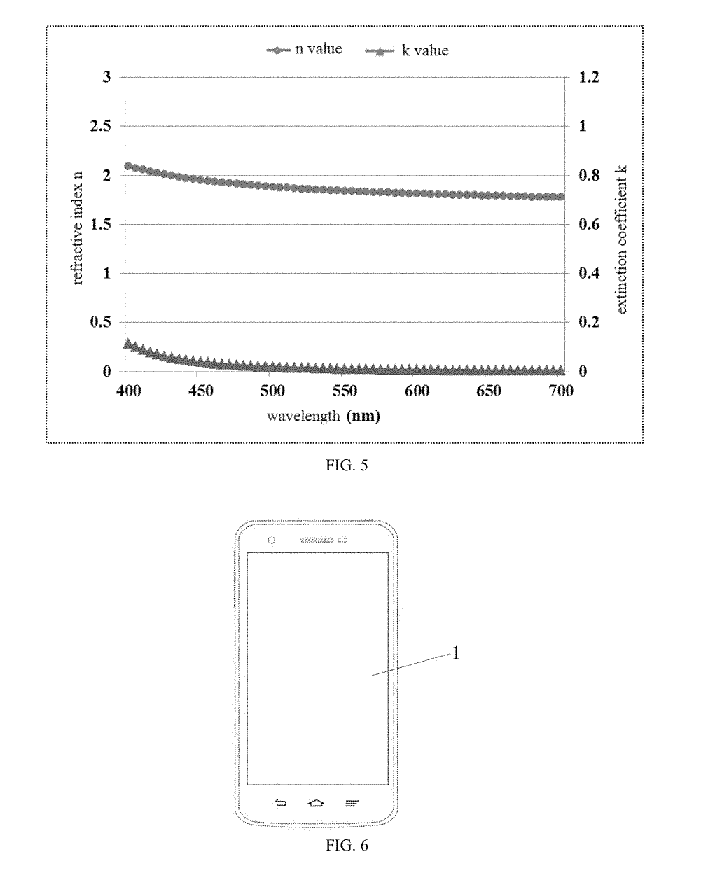

As shown in FIGS. 3-5, FIG. 3 is a graph showing refractive index and extinction coefficient of CP4; FIG. 4 is a graph showing refractive index and extinction coefficient of CP11; FIG. 5 is a graph showing refractive index and extinction coefficient of Com-1 (CBP). As can be seen from FIG. 3-5, for light having a wavelength in the range of 400 nm to 700 nm, the nitrogen heterocycle-fused benzene-benzimidazole organic compounds provided by the present disclosure have a refractive index greater than or equal to 2.0. However, for light having a wavelength in the range of 430 nm to 700 nm, the refractive index of the Comparative Example Com-1 is less than 2.0. Moreover, the value of the extinction coefficient k of the nitrogen heterocycle-fused benzene-benzimidazole organic compound designed in the present disclosure is almost 0 for light having a wavelength greater than 450 nm of the blue light wavelength, which will not affect luminescence of the material of the light-emitting layer in the region of blue light.

Organic Light-Emitting Device

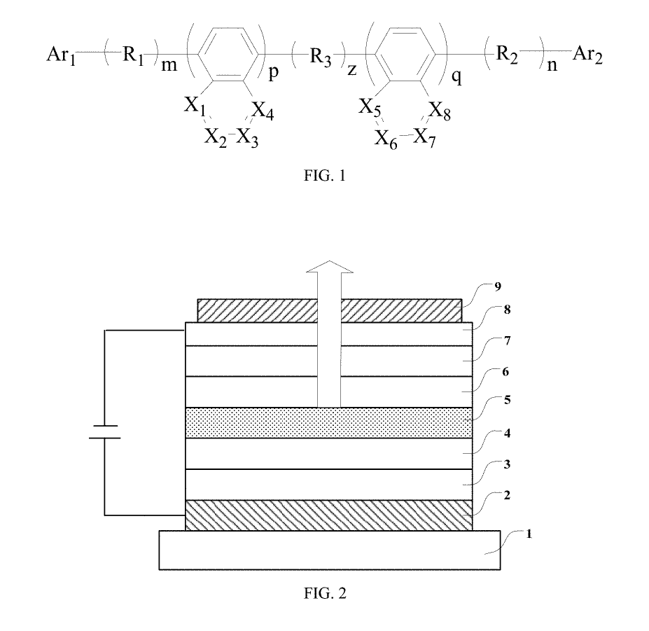

FIG. 2 is a structural schematic diagram showing an organic light-emitting device according to an embodiment of the present disclosure. As shown in FIG. 2, the organic light-emitting device provided by the present disclosure includes: a substrate 1, an ITO anode 2, a first hole transmission layer 3, a second hole transmission layer 4, a light-emitting layer 5, a first electron transmission layer 6, a second electron transmission layer 7, a cathode 8 (magnesium silver electrode, mass ratio 9:1 of magnesium to silver) and a capping layer CPL9. The ITO anode 2 has a thickness of 15 nm. The first hole transmission layer 3 has a thickness of 10 nm. The second hole transmission layer 4 has a thickness of 110 nm. The light-emitting layer 5 has a thickness of 30 nm. The first electron transmission layer 6 has a thickness of 30 nm. The second electron transmission layer 7 has a thickness of 5 nm. The magnesium silver electrode 8 has a thickness of 15 nm. The capping layer CPL9 has a thickness of 100 nm.

The steps for preparing the organic light-emitting device according to the present disclosure are as follows.

1) A glass substrate 1 was cut into a size of 50 mm×50 mm×0.7 mm, subjected to ultrasonic treatment respectively in isopropyl alcohol and in deionized water for 30 minutes, and then exposed to ozone for about 10 minutes for cleaning. The obtained glass substrate with an ITO anode 2 is mounted on a vacuum deposition apparatus.

2) On the ITO anode 2, the material HAT-CN was evaporated by vacuum evaporation to a thickness of 10 nm and used as the first hole transmission layer 3.

3) The material TAPC was evaporated by vacuum evaporation on the first hole transmission layer 3 to a thickness of 110 nm and used as the second hole transmission layer 4.

4) The light-emitting layer 5 was co-deposited on the second hole transmission layer 4 in which CBP is used as a host material. Ir(ppy)3 is used as a doping material, a mass ratio of Ir(ppy)3 to CBP is 1:9. The light-emitting layer 5 has a thickness of 30 nm.

5) The material TPBI was evaporated by vacuum evaporation on the light-emitting layer 5 to a thickness of 30 nm and used as the first electron transmission layer 6.

6) The material Alq3 was evaporated by vacuum evaporation on the first electron transmission layer 6 to a thickness of 5 nm and used as the second electron transmission layer 7.

7) Magnesium silver electrode was formed by vacuum evaporation on the second electron transmission layer 7 to a thickness of 15 nm and used as the cathode 8, in which the mass ratio of Mg to Ag is 9:1.

8) The compound CP1 according to the present disclosure was evaporated by vacuum evaporation on the cathode 8 to a thickness of 100 nm and used as a capping layer CPL.

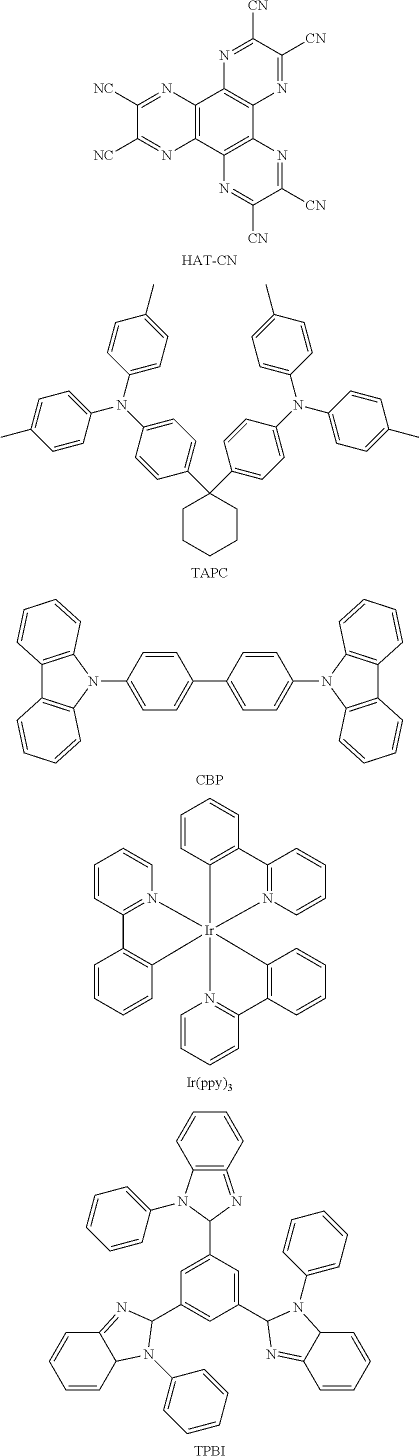

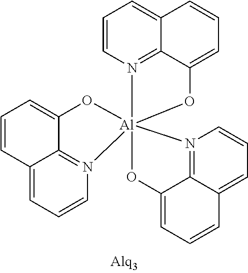

The preparation methods of the device examples 2-12 and the device comparative example 1 were the same except that the compounds used in the vacuum evaporation at step 7) are different. The compounds used in the step 7) of the device examples 2-12 and the device comparative example 1 are CP4, CP6, CP11, CP12, CP15, CP18, CP23, CP24, CP27, CP30, CP32, and CBP, respectively. All of the devices prepared above differ only in the material selection of the CPL layer, and the materials of other layers, for example, the light-emitting layer and the auxiliary layers, are the same.

TABLE 3
test results of luminescence performance of the devices

| | | @10 mA/cm² | | service life |
|---|---|---|---|---|
| | | current | | (based on |
| | drive | efficiency | brightness | comparative |
| No. | voltage(V) | (cd/A) | (cd/m²) | example 1) |
| device example 1 | 4.3 | 36.5 | 3651.4 | 1.9 |
| device example 2 | 4.5 | 35.4 | 3539.6 | 1.8 |
| device example 3 | 4.4 | 36.1 | 3609.5 | 2.2 |
| device example 4 | 4.2 | 35.8 | 3581.7 | 2.1 |
| device example 5 | 4.3 | 36.2 | 3621.8 | 1.7 |
| device example 6 | 4.4 | 35.4 | 3542.1 | 1.6 |
| device example 7 | 4.5 | 35.7 | 3570.4 | 2.0 |
| device example 8 | 4.3 | 36.3 | 3631.2 | 2.1 |
| device example 9 | 4.4 | 36.6 | 3659.4 | 1.9 |
| device example 10 | 4.3 | 36.2 | 3620.9 | 2.1 |
| device example 11 | 4.4 | 35.8 | 3581.8 | 1.9 |
| device example 12 | 4.3 | 36.7 | 3669.8 | 1.8 |
| device comparative | 5.0 | 26.9 | 2691.7 | 1 |
| example 1 | | | | |

As can be seen from the above Table 3, the optical devices using the nitrogen heterocycle-fused benzene-benzimidazole organic compound according to the present disclosure have a lower driving voltage, higher current efficiency, higher brightness, and longer service life compared with the device comparative example 1.

Another embodiment of the present disclosure provides a display panel including the organic light-emitting device as described above.

Yet another embodiment of the present disclosure provides a display apparatus including the display panel as described above.

In the present disclosure, the organic light-emitting device may be an OLED, which can be used in an organic light-emitting display apparatus. The organic light-emitting display apparatus may be a mobile phone display screen, a computer display screen, a liquid crystal television display screen, a smart watch display screen, or a smart car display panel, VR or AR helmet display screen, and display screens of various smart devices, etc. FIG. 6 is a schematic diagram showing a display apparatus according to an embodiment of the present disclosure, in which the reference number 1 is a mobile phone display screen.

What is claimed is:

1. A nitrogen heterocycle-fused benzene-benzimidazole organic compound, having a chemical structure represented by chemical formula I: 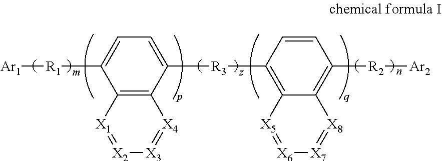
wherein each of X₁-X₈ independently represents a nitrogen atom or a carbon atom, and at least one of X₁-X₄ is a nitrogen atom, and at least one of X₅-X₈ is a nitrogen atom; m, n, p, q, and z each independently represent a number of 0, 1, or 2, and p+q≥1;
R₁, R₂, and R₃ each independently represent a single bond, substituted or unsubstituted C1-C30 alkyl, substituted or unsubstituted silicylene, substituted or unsubstituted C1-C30 alkoxy, substituted or unsubstituted C6-C40 aryl, substituted or unsubstituted C10-C40 fused aryl, or substituted or unsubstituted C6-C40 heteroaryl;
Ar₁ and Ar₂ each independently represent one of the following structures shown by chemical formula II, chemical formula III, and chemical formula IV: 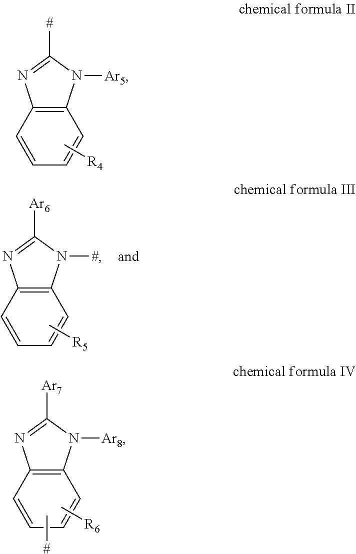
wherein # represents a bonding position in the chemical formula I;
Ar₅, Ar₆ and Ar₇ each are independently selected from a group consisting of hydrogen atom, substituted or unsubstituted C1-C30 alkyl, substituted or unsubstituted silicylene, substituted or unsubstituted C1-C30 alkoxy, substituted or unsubstituted C6-C40 aryl, substituted or unsubstituted C10-C40 fused aryl, and substituted or unsubstituted C6-C40 heteroaryl; and
R₄, R₅, and R₆ each are independently selected from a group consisting of hydrogen atom, substituted or unsubstituted C1-C30 alkyl, substituted or unsubstituted silicylene, substituted or unsubstituted C1-C30 alkoxy, substituted or unsubstituted C6-C40 aryl, substituted or unsubstituted C10-C40 fused aryl, and substituted or unsubstituted C6-C40 heteroaryl.

2. The nitrogen heterocycle-fused benzene-benzimidazole organic compound according to claim 1, wherein in the chemical formula I, m=n=0, p>0, q>0, and z>0.

3. The nitrogen heterocycle-fused benzene-benzimidazole organic compound according to claim 1, wherein in the chemical formula I, q=z=0.

4. The nitrogen heterocycle-fused benzene-benzimidazole organic compound according to claim 1, wherein in the chemical formula I, m>0, and n=0; or, n>0, and m=0.

5. The nitrogen heterocycle-fused benzene-benzimidazole organic compound according to claim 1, wherein in the chemical formula I, p>0, q>0, and m=n=z=0.

6. The nitrogen heterocycle-fused benzene-benzimidazole organic compound according to claim 1, wherein in the chemical formula I, p>0, and m=n=q=z=0.

7. The nitrogen heterocycle-fused benzene-benzimidazole organic compound according to claim 1, wherein in the chemical formula I, m=n.

8. The nitrogen heterocycle-fused benzene-benzimidazole organic compound according to claim 1, wherein in the chemical formula I, p>0, q>0, and
X₁ and X₈ represent a same atom, X₂ and X₇ represent a same atom, X₃ and X₆ represent a same atom, and X₄ and X₅ represent a same atom.

9. The nitrogen heterocycle-fused benzene-benzimidazole organic compound according to claim 1, wherein in the chemical formula I, R₁ and R₂ represent a same substituent.

10. The nitrogen heterocycle-fused benzene-benzimidazole organic compound according to claim 1, wherein in the chemical formula I, Ar₁ and Ar₂ represent a same substituent.

11. The nitrogen heterocycle-fused benzene-benzimidazole organic compound according to claim 1, wherein in the chemical formula I, only one of X₁-X₄ represents a nitrogen atom, and/or only one of X₅-X₈ represents a nitrogen atom.

12. The nitrogen heterocycle-fused benzene-benzimidazole organic compound according to claim 1, wherein in the chemical formula I, at least two of X₁-X₄ represent a nitrogen atom, and/or, at least two of X₅-X₈ represent a nitrogen atom.

13. The nitrogen heterocycle-fused benzene-benzimidazole organic compound according to claim 1, wherein the nitrogen heterocycle-fused benzene-benzimidazole organic compound is one of the following compounds:

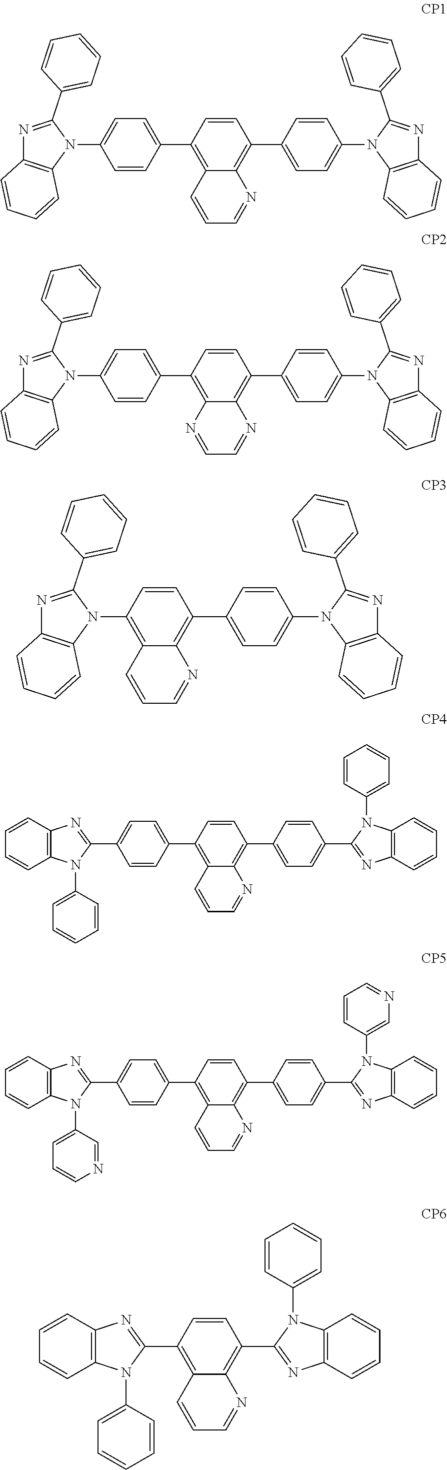

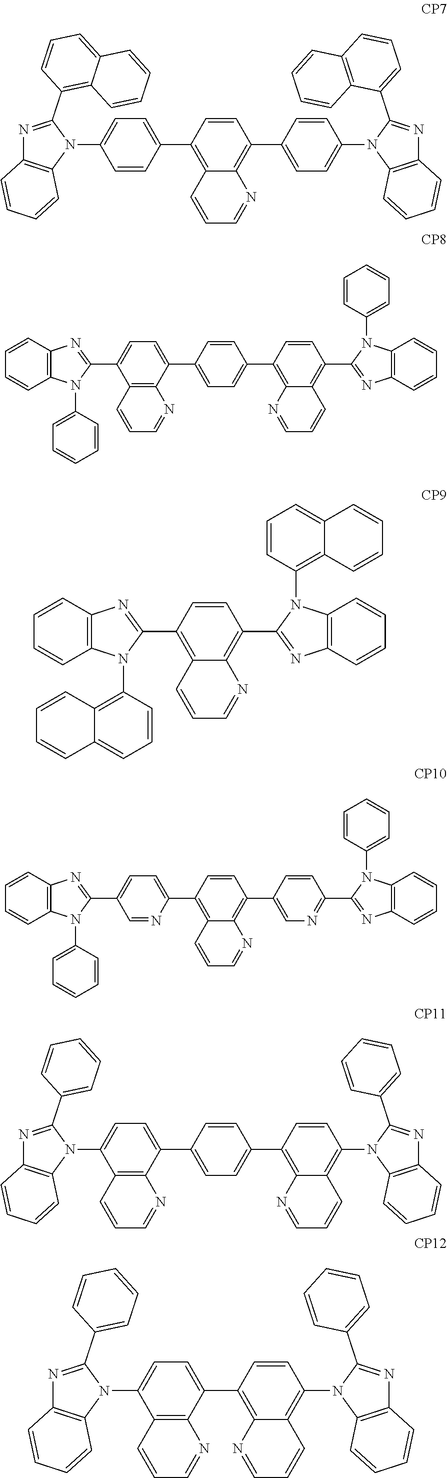

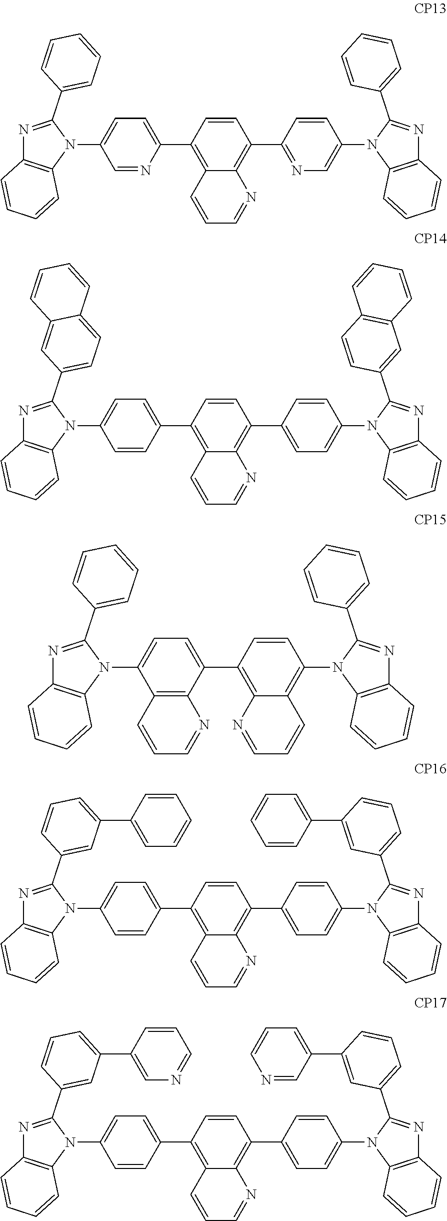

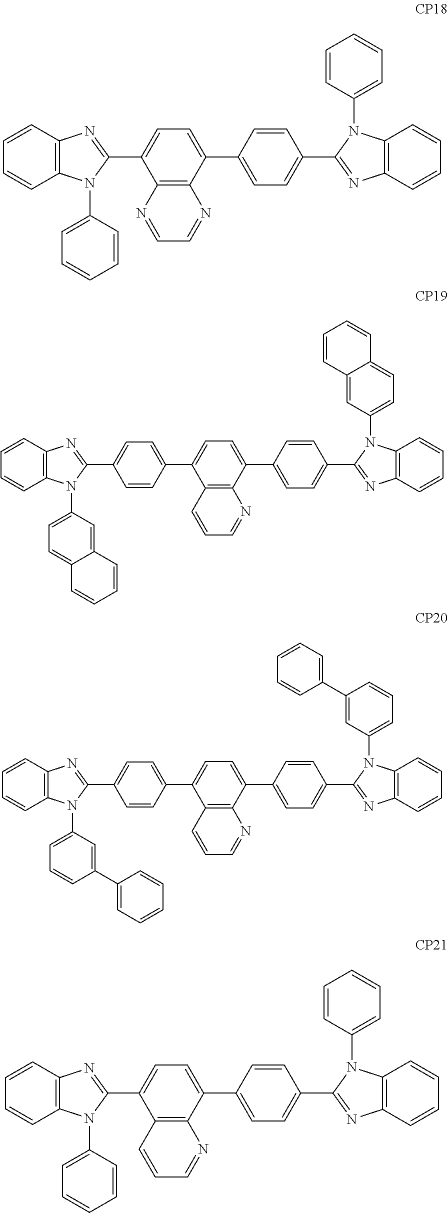 [SMILES: c1ccc(-n2c(-c3ccc(-c4ccc(-c5nc6ccccc6n5-c5ccccc5)c5nccnc45)cc3)nc3ccccc32)cc1 ; c1ccc2cc(-n3c(-c4ccc(-c5ccc(-c6ccc(-c7nc8ccccc8n7-c7ccc8ccccc8c7)cc6)c6ncccc56)cc4)nc4ccccc43)ccc2c1 ; c1ccc(-c2cccc(-n3c(-c4ccc(-c5ccc(-c6ccc(-c7nc8ccccc8n7-c7cccc(-c8ccccc8)c7)cc6)c6ncccc56)cc4)nc4ccccc43)c2)cc1 ; c1ccc(-n2c(-c3ccc(-c4ccc(-c5nc6ccccc6n5-c5ccccc5)c5cccnc45)cc3)nc3ccccc32)cc1]

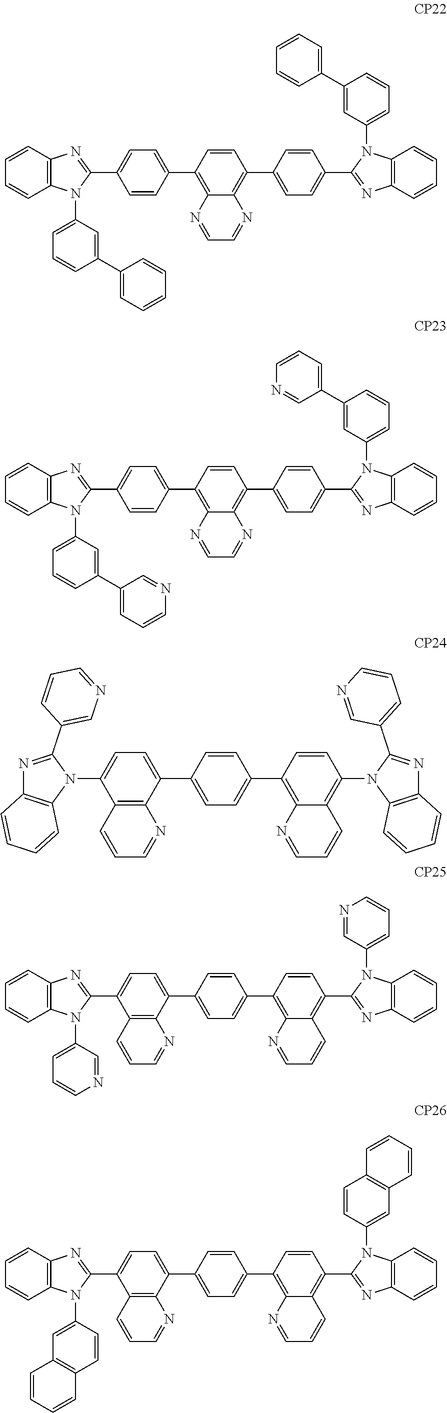

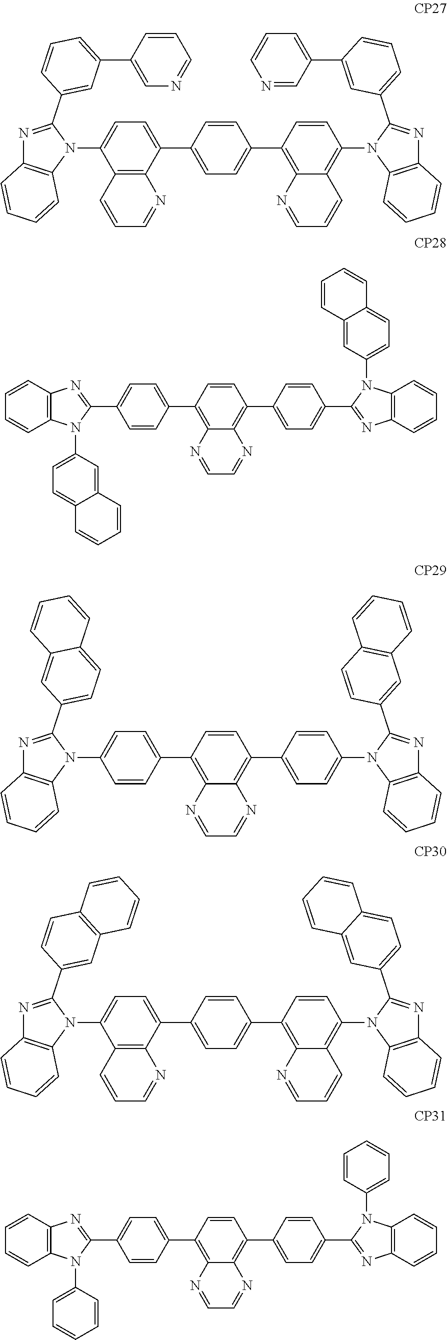

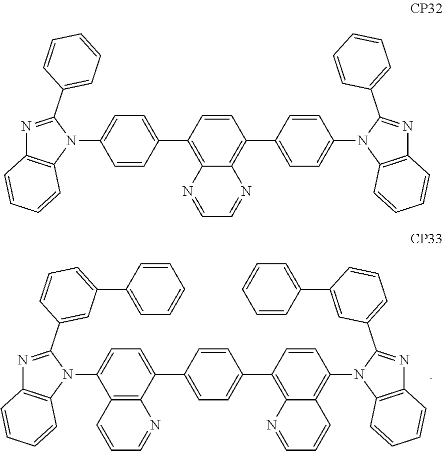 [SMILES: c1ccc(-c2nc3ccccc3n2-c2ccc(-c3ccc(-c4ccc(-n5c(-c6ccccc6)nc6ccccc65)cc4)c4nccnc34)cc2)cc1 ; c1ccc(-c2cccc(-c3nc4ccccc4n3-c3ccc(-c4ccc(-c5ccc(-n6c(-c7cccc(-c8ccccc8)c7)nc7ccccc76)c6cccnc56)cc4)c4ncccc34)c2)cc1]

14. The nitrogen heterocycle-fused benzene-benzimidazole organic compound according to claim 1, wherein, for visible light having a wavelength between 400 nm and 700 nm, the nitrogen heterocycle-fused benzene-benzimidazole organic compound has a refractive index n greater than or equal to 2.0.

15. The nitrogen heterocycle-fused benzene-benzimidazole organic compound according to claim 1, wherein, for visible light having a wavelength between 430 nm and 700 nm, the nitrogen heterocycle-fused benzene-benzimidazole organic compound has an extinction coefficient k less than or equal to 0.0.

16. The nitrogen heterocycle-fused benzene-benzimidazole organic compound according to claim 1, wherein, for visible light having a wavelength between 400 nm and 700 nm, the nitrogen heterocycle-fused benzene-benzimidazole organic compound has a transmittance greater than 65%.

17. A display panel, comprising an organic light-emitting device, wherein the organic light-emitting device comprises an anode, a cathode arranged opposite to the anode, a capping layer located at a side of the cathode facing away from the anode, and an organic layer located between the anode and the cathode, wherein the organic layer comprises a hole transmission layer, an electron transmission layer, and a light-emitting layer, at least one of the capping layer, the hole transmission layer, the electron transmission layer, and the light-emitting layer is made of a nitrogen heterocycle-fused benzene-benzimidazole organic compound, having a chemical structure represented by chemical formula I: 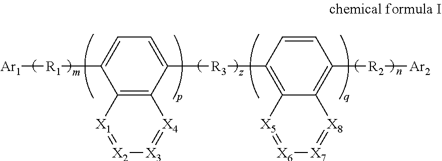
wherein each of X₁-X₈ independently represents a nitrogen atom or a carbon atom, and at least one of X₁-X₄ is a nitrogen atom, and at least one of X₅-X₈ is a nitrogen atom; m, n, p, q, and z each independently represent a number of 0, 1, or 2, and p+q≥1;
R₁, R₂, and R₃ each independently represent a single bond, substituted or unsubstituted C1-C30 alkyl, substituted or unsubstituted silicylene, substituted or unsubstituted C1-C30 alkoxy, substituted or unsubstituted C6-C40 aryl, substituted or unsubstituted C10-C40 fused aryl, or substituted or unsubstituted C6-C40 heteroaryl;
Ar₁ and Ar₂ each independently represent one of the following structures shown by chemical formula II, chemical formula III, and chemical formula IV: 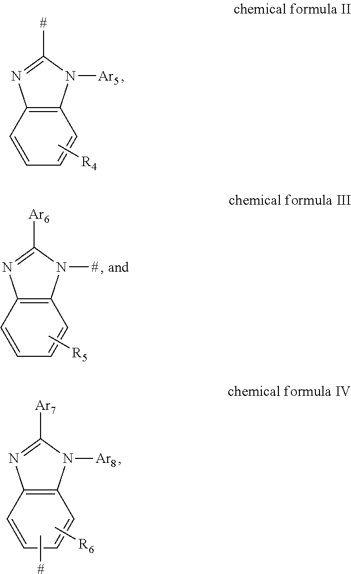
wherein # represents a bonding position in the chemical formula I;
Ar₅, Ar₆ and Ar₇ each are independently selected from a group consisting of hydrogen atom, substituted or unsubstituted C1-C30 alkyl, substituted or unsubstituted silicylene, substituted or unsubstituted C1-C30 alkoxy, substituted or unsubstituted C6-C40 aryl, substituted or unsubstituted C10-C40 fused aryl, and substituted or unsubstituted C6-C40 heteroaryl; and
R₄, R₅, and R₆ each are independently selected from a group consisting of hydrogen atom, substituted or unsubstituted C1-C30 alkyl, substituted or unsubstituted silicylene, substituted or unsubstituted C1-C30 alkoxy, substituted or unsubstituted C6-C40 aryl, substituted or unsubstituted C10-C40 fused aryl, and substituted or unsubstituted C6-C40 heteroaryl.

18. A display apparatus comprising a display panel, wherein the display panel comprises an organic light-emitting device, wherein the organic light-emitting device comprises an anode, a cathode arranged opposite to the anode, a capping layer located at a side of the cathode facing away from the anode, and an organic layer located between the anode and the cathode, wherein the organic layer comprises a hole transmission layer, an electron transmission layer, and a light-emitting layer, at least one of the capping layer, the hole transmission layer, the electron transmission layer, and the light-emitting layer is made of a nitrogen heterocycle-fused benzene-benzimidazole organic compound, having a chemical structure represented by chemical formula I: 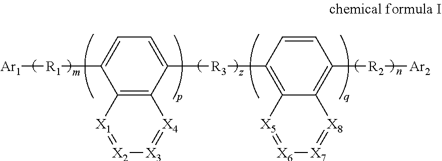
wherein each of X₁-X₈ independently represents a nitrogen atom or a carbon atom, and at least one of X₁-X₄ is a nitrogen atom, and at least one of X₅-X₈ is a nitrogen atom; m, n, p, q, and z each independently represent a number of 0, 1, or 2, and p+q≥1;
R₁, R₂, and R₃ each independently represent a single bond, substituted or unsubstituted C1-C30 alkyl, substituted or unsubstituted silicylene, substituted or unsubstituted C1-C30 alkoxy, substituted or unsubstituted C6-C40 aryl, substituted or unsubstituted C10-C40 fused aryl, or substituted or unsubstituted C6-C40 heteroaryl;
Ar₁ and Ar₂ each independently represent one of the following structures shown by chemical formula II, chemical formula III, and chemical formula IV: 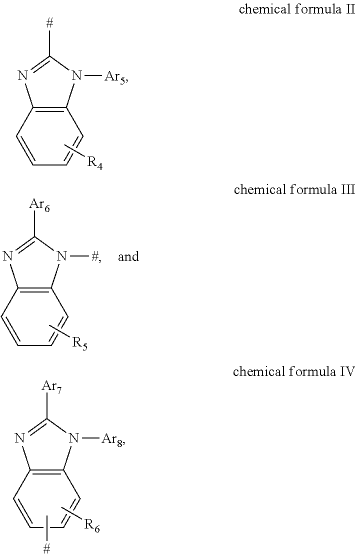
wherein # represents a bonding position in the chemical formula I;
Ar₅, Ar₆ and Ar₇ each are independently selected from a group consisting of hydrogen atom, substituted or unsubstituted C1-C30 alkyl, substituted or unsubstituted silicylene, substituted or unsubstituted C1-C30 alkoxy, substituted or unsubstituted C6-C40 aryl, substituted or unsubstituted C10-C40 fused aryl, and substituted or unsubstituted C6-C40 heteroaryl; and
R₄, R₅, and R₆ each are independently selected from a group consisting of hydrogen atom, substituted or unsubstituted C1-C30 alkyl, substituted or unsubstituted silicylene, substituted or unsubstituted C1-C30 alkoxy, substituted or unsubstituted C6-C40 aryl, substituted or unsubstituted C10-C40 fused aryl, and substituted or unsubstituted C6-C40 heteroaryl.
